(19) United States
(12) Patent Application Publication
(10) Pub. No.: US 2026/0213001 A1
(43) Pub. Date: Jul. 23, 2026

(54) COMPUTER-IMPLEMENTED METHOD AND SYSTEM FOR DETERMINING URGENCY LEVEL FOR TREATMENT OF USER

(71) Applicant: NEC Corporation, Tokyo (JP)

(72) Inventors: Charles Chi Hin CHOY, Singapore (SG); Takuya HIRAOKA, Tokyo (JP); Ryoma OAMI, Tokyo (JP); Ni Ni SOE, Singapore (SG)

(73) Assignee: NEC Corporation, Tokyo (JP)

(21) Appl. No.: 19/443,089

(22) Filed: Jan. 8, 2026

(30) Foreign Application Priority Data
Jan. 21, 2025 (SG) 10202500184W

(51) Int. Cl. G16H 40/20; G06F 16/3329

(52) U.S. Cl. CPC G16H 40/20; G06F 16/33295

(57) ABSTRACT

The present disclosure provides a method and an apparatus for determining an urgency level for treatment of a user, the method comprising: verifying, by a processor, whether there is an inconsistency in an argument set comprising a plurality of arguments, each of the plurality of arguments being associated with an urgency level that is based on a condition of a user and a rationale for the urgency level; and determining, by the processor, the urgency level for treatment of the user based on the argument set and the verification.

### INCORPORATION BY REFERENCE

This application is based upon and claims the benefit of priority from Singapore Patent Application No. 10202500184W, filed on Jan. 21, 2025, the disclosure of which is incorporated herein in its entirety by reference.

### TECHNICAL FIELD

The present disclosure relates broadly, but not exclusively, to a method and system for determining an urgency level for treatment of a user.

### BACKGROUND ART

Traditional triage systems primarily rely on verbal question and answer (Q&A) and patient-reported symptoms but fail to capture non-verbal cues such as facial expressions and speech emotions, which are often crucial in determining patient severity. Human clinicians, especially nurses, naturally observe patients'stress, pain, and discomfort by interpreting non-verbal behaviours. Such subtle signs of distress often indicate a more severe condition. The lack of such insights may lead to under-prioritisation of patients.

Further, even in a case where capturing such non-verbal cues, current triage systems are ill-equipped with handling conflicting triage results (e.g., different levels of urgency being presented for different sources of triage systems utilizing verbal Q&A or non-verbal cues to determine an urgency level for treatment of a user), resulting in unreliable and inaccurate levels of urgency for treatment being determined for a patient.

Furthermore, a patient's symptoms and conditions can evolve rapidly. In cases where an initial assessment is inconclusive or where symptoms worsen, it is crucial to capture new information and re-evaluate. Existing systems lack an iterative triage process that dynamically adjusts to capture ongoing changes and integrate prior data for a refined understanding of the patient's condition.

Herein disclosed are embodiments of a method and system for determining an urgency level for treatment of a user that addresses one or more of the above problems.

Furthermore, other desirable features and characteristics will become apparent from the subsequent detailed description and the appended claims, taken in conjunction with the accompanying drawings and this background of the disclosure.

### SUMMARY

In an example first aspect, the present disclosure provides a method for determining an urgency level for treatment of a user, comprising: verifying, by a processor, whether there is an inconsistency in an argument set comprising a plurality of arguments, each of the plurality of arguments being associated with an urgency level that is based on a condition of a user and a rationale for the urgency level; and determining, by the processor, the urgency level for treatment of the user based on the argument set and the verification.

In an example second aspect, the present disclosure provides a system for determining an urgency level for treatment of a user, comprising: at least one processor; and at least one memory including computer program code; the at least one memory and the computer program code configured to, with at least one processor, cause the system at least to: verify whether there is an inconsistency in an argument set comprising a plurality of arguments, each of the plurality of arguments being associated with an urgency level that is based on a condition of a user and a rationale for the urgency level; and determine the urgency level for treatment of the user based on the argument set and the verification.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

The accompanying Figures, where like reference numerals refer to identical or functionally similar elements throughout the separate views and which together with the detailed description below are incorporated in and form part of the specification, serve to illustrate various embodiments and to explain various principles and advantages in accordance with a present embodiment, by way of non-limiting example only.

Embodiments of the disclosure will be better understood and readily apparent to one of ordinary skill in the art from the following written description, by way of example only, and in conjunction with the drawings, in which:

FIG. 1 shows a block diagram for a system for determining an urgency level for treatment of a user according to various embodiments of the present disclosure.

FIG. 2 shows a block diagram for a patient console according to various embodiments of the present disclosure.

FIG. 3 shows a block diagram for external systems according to various embodiments of the present disclosure.

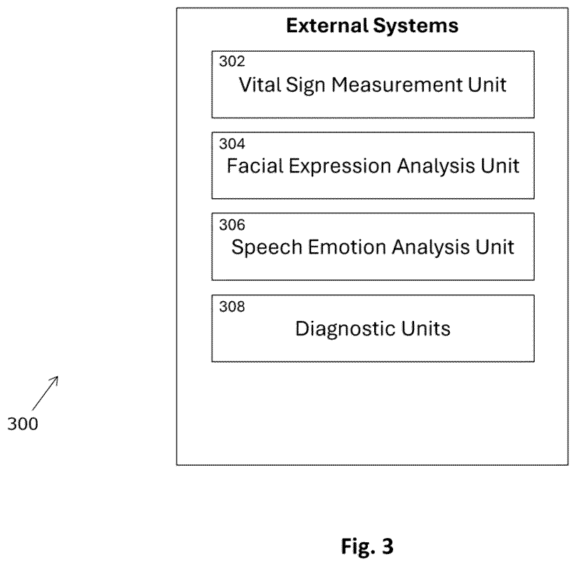

FIG. 4 shows a block diagram for a triaging core according to various embodiments of the present disclosure.

FIG. 5 shows a block diagram for a clinician console according to various embodiments of the present disclosure.

FIG. 6 shows an exemplary flow diagram for determining an urgency level for treatment of a user according to various embodiments of the present disclosure.

FIG. 7 shows an exemplary flow diagram for how conflicts among different urgency level results may be resolved according to various embodiments of the present disclosure.

FIG. 8 shows an exemplary illustration of an urgency level based on an electronic medical record (EMR) of a user according to an embodiment of the present disclosure.

FIG. 9 shows an exemplary illustration of an urgency level based on vital signs monitoring of a user according to an embodiment of the present disclosure.

FIG. 10 shows an exemplary illustration of an urgency level based on image recognition of a user according to an embodiment of the present disclosure.

FIG. 11 shows an exemplary illustration of an urgency level based on a question and answer (Q&A) session between a dynamic questioning agent and a user according to an embodiment of the present disclosure.

FIG. 12 shows an exemplary illustration of a conflict resolution process for different urgency level results according to an embodiment of the present disclosure.

FIG. 13 shows an exemplary illustration of a conflict resolution proposal according to an embodiment of the present disclosure.

FIG. 14 shows an exemplary illustration of how the conflict resolution proposal may be responded to according to an embodiment of the present disclosure.

FIG. 15 shows an exemplary illustration of an updated conflict resolution process as a result of the conflict resolution proposal according to an embodiment of the present disclosure.

FIG. 16 shows an exemplary illustration of an alternative conflict resolution proposal according to an embodiment of the present disclosure.

FIG. 17 shows an exemplary illustration of how the alternative conflict resolution proposal may be responded to according to an embodiment of the present disclosure.

FIG. 18 shows an exemplary illustration depicting an updated urgency level for image recognition of the user according to an embodiment of the present disclosure.

FIG. 19 shows an exemplary illustration of an updated conflict resolution process as a result of the alternative conflict resolution proposal according to an embodiment of the present disclosure.

FIG. 20 shows another exemplary illustration of how the further alternative conflict resolution proposal may be responded to according to an embodiment of the present disclosure.

FIG. 21 shows an exemplary illustration depicting a newly added urgency level based on an electrocardiogram (ECG) result of the user according to an embodiment of the present disclosure.

FIG. 22 shows another exemplary illustration of an updated conflict resolution process as a result of the alternative conflict resolution proposal according to an embodiment of the present disclosure.

FIG. 23 shows a flow diagram for determining an urgency level for treatment of a user according to various embodiments of the present disclosure.

FIG. 24 shows a schematic diagram of an exemplary computing device suitable for use to execute the method in FIGS. 6 to 23.

Skilled artisans will appreciate that elements in the figures are illustrated for simplicity and clarity and have not necessarily been depicted to scale. For example, the dimensions of some of the elements in the illustrations, block diagrams or flowcharts may be exaggerated in respect to other elements to help to improve understanding of the present embodiments.

### EXAMPLE EMBODIMENTS

### Terms Description

An urgency level refers to a measure of priority for treatment of a user based on a triage process of assessing the user's status and a severity of a condition that the user may have. One or more urgency levels (e.g., a first, second, third, or more urgency levels) may be obtained from one or more triage agents (e.g., a program, computer device, machine, or other similar entity that may determine an urgency level based on a condition of a user that may be assessed from corresponding input from the user or data associated with the user) utilizing one or more triage processes, for example based on an electronic medical record (EMR) of a user, a physiological condition of the user, an electrocardiogram (ECG) of the user, an input of the user in response to a question (for example through a Q&A session with the user), or other similar processes. A conflict resolution process may be performed with the one or more urgency levels in order to reach a resolution for conflicting urgency levels obtained from different triage processes, and determine an appropriate urgency level for the user. This urgency level may thus serve as an indicator for, for example, a medical institution such as a clinic or hospital to queue the user appropriately among other users who need to seek treatment for their condition(s). In the present disclosure, “urgency level” and “triage result” may be used interchangeably. An argument refers to a pair of an urgency level and its rationale that is a basis for determining the urgency level. It will be appreciated that the input and data to be processed by a system for determining an urgency level for treatment of a user are obtained from one or more devices, modules, consoles, servers, or other similar hardware that may be configured to provide such input and data with or without interaction with the user.

A user refers to a person, for example a patient or other similar entity undergoing a triage process to determine an urgency level for treatment of the user. In the present disclosure, ‘user’ and ‘patient’ may be used interchangeably. It will be appreciated that a user may also refer to an entity that is providing data and urgency levels (that may be simulated or real) to the system to simulate, train and/or test the system for determining an urgency level.

A condition of a user refers to a status of the user or a symptom experienced by the user based on a triage processing of input and/or data received from the user. For example, the condition may be based on an electronic medical record (EMR) of a user, a physiological condition of the user, an electrocardiogram (ECG) of the user, a summary of symptoms generated from an input/inputs of the user in response to a question (for example through a Q&A session with the user), or other similar processes. A corresponding urgency level for treatment of the user may be determined through a triage process for each condition, for example by processing the data associated with the respective condition based on historical data indicating a corresponding urgency level for a condition. It will be appreciated that the input and data to be processed by the system for determining an urgency level for treatment of a user are obtained from one or more devices, modules, consoles, servers, or other similar hardware that may be configured to provide such input and data with or without interaction with the user.

An inconsistency in an urgency level refers to a finding that the urgency level does not match with another urgency level that is reached via another triage process for a same or a different condition of the user, or other similar conflict. For example, an inconsistency between a first urgency level (determined based on a first condition of a user) and a second urgency level (determined based on a second condition of the user) may be verified by processing the first urgency level, second urgency level, the first condition and the second condition based on past data, such as historical data indicating a corresponding urgency level for a condition (e.g., a conflict resolution process). The processing may be performed by a processor utilizing a model such as a machine learning (ML) model, large language model (LLM), artificial intelligence (AI), rule-based approach (e.g., based on one or more rules that may be set in the utilized model by a medical institution or expert, a ML model, LLM, AI, or other similar entity), or other similar model. If it is verified that there is an inconsistency, the inconsistency may be resolved via a conflict resolution proposal that may be generated after the verification. For example, the conflict resolution proposal may be to include an additional urgency level for further processing. The additional urgency level may be one that is determined for a same condition as an urgency level that was regarded as inconsistent by the conflict resolution proposal, but through a triage process that may be different from that used for the inconsistent urgency level. In another example, the conflict resolution proposal may be to review and, if required, update one or more urgency levels (e.g., one or more urgency levels that are verified by the conflict resolution process as inconsistent, or for all urgency levels that were processed) based on the review. The review may be based on further data that is obtained in response to the conflict resolution proposal for reviewing and/or updating the inconsistency urgency level.

Embodiments of the present disclosure will be described, by way of example only, with reference to the drawings. Like reference numbers and characters in the drawings refer to like elements or equivalents.

Some portions of the description which follows are explicitly or implicitly presented in terms of algorithms and functional or symbolic representations of operations on data within a computer memory. These algorithmic descriptions and functional or symbolic representations are the means used by those skilled in the data processing arts to convey most effectively the substance of their work to others skilled in the art. An algorithm is here, and generally, conceived to be a self-consistent sequence of steps leading to a desired result. The steps are those requiring physical manipulations of physical quantities, such as electrical, magnetic or optical signals capable of being stored, transferred, combined, compared, and otherwise manipulated.

Unless specifically stated otherwise, and as apparent from the following, it will be appreciated that throughout the present specification, discussions utilizing terms such as “verifying”, “determining”, “processing”, “updating”, “calculating”, “generating”, “initializing”, “outputting”, “receiving”, “retrieving”, “transmitting”, “identifying” or the like, refer to the action and processes of a computer system, or similar electronic device, that manipulates and transforms data represented as physical quantities within the computer system into other data similarly represented as physical quantities within the computer system or other information storage, transmission or display devices.

The present specification also discloses apparatus for performing the operations of the methods. Such apparatus may be specially constructed for the required purposes, or may comprise a computer or other device selectively activated or reconfigured by a computer program stored in the computer. The algorithms and displays presented herein are not inherently related to any particular computer or other apparatus. Various machines may be used with programs in accordance with the teachings herein. Alternatively, the construction of more specialized apparatus to perform the required method steps may be appropriate. The structure of a computer will appear from the description below.

In addition, the present specification also implicitly discloses a computer program, in that it would be apparent to the person skilled in the art that the individual steps of the method described herein may be put into effect by computer code. The computer program is not intended to be limited to any particular programming language and implementation thereof. It will be appreciated that a variety of programming languages and coding thereof may be used to implement the teachings of the disclosure contained herein. Moreover, the computer program is not intended to be limited to any particular control flow. There are many other variants of the computer program, which can use different control flows without departing from the spirit or scope of the disclosure.

Furthermore, one or more of the steps of the computer program may be performed in parallel rather than sequentially. Such a computer program may be stored on any computer readable medium. The computer readable medium may include storage devices such as magnetic or optical disks, memory chips, or other storage devices suitable for interfacing with a computer. The computer readable medium may also include a hard-wired medium such as exemplified in the Internet system, or wireless medium such as exemplified in the GSM mobile telephone system. The computer program in a case where it is loaded and executed on such a computer effectively results in an apparatus that implements the steps of the preferred method.

### Exemplary Embodiments

Various embodiments of the present disclosure relate to a method and an apparatus for determining an urgency level for treatment of a user.

The proposed solution solves the problem of incomplete and inconsistent triage decisions by integrating multimodal data such as voice, text, and vital signs to mimic how human clinicians assess severity of a condition of a user. Unlike traditional systems that typically rely solely on verbal Q&A, the proposed system processes non-verbal cues—like facial expressions and tone of voice—using advanced speech recognition, facial analysis, and remote vital sign monitoring. By dynamically weighing and fusing these inputs, it advantageously replicates a clinician's ability to detect subtle signs of pain, stress, or distress, ensuring that critical symptoms are not overlooked. This leads to more accurate, real-time triage recommendations, improving patient prioritization and care outcomes.

It also solves the problem of non-adaptive triaging rules which cannot address changing patients'conditions by using insights from previous rounds of triage to adapt its questioning approach based on past triage results, sensor data, and EMR-based medical history. This iterative capability advantageously allows the proposed system to refine its assessment in each subsequent round, clarifying evolving or ambiguous conditions of a user and capturing any newly reported changes associated with the user's conditions. This improves confidence in the triage result, reduces redundant Q&A, while ensuring that the system's understanding of the user's condition is continuously updated and complete.

In an implementation, the proposed system collects and processes multiple data streams—such as vital signs, symptoms, facial expressions, speech emotion, and medical history—in real time. This holistic approach enables a comprehensive understanding of a user's condition, allowing for quicker and more accurate triage decisions based on multiple simultaneous inputs. In a case where inconsistencies arise between multimodal triage results, the system leverages an argumentation framework to resolve conflicts. Advantageously, a conflict resolver identifies the most credible triage result by systematically analyzing and resolving inconsistencies. Further, a dynamic questioning agent may be configured to tailor its questioning based on insights from prior triage results and sensor data collected in previous rounds. The dynamic questioning agent may be a software, application, or other similar media that is used to facilitate a Q&A session with a user, in which it may propose questions to ascertain a condition of a user and determine an urgency level based on the ascertained condition. In an implementation, the dynamic questioning agent may utilize a model such as a ML model, LLM, AI, rule-based approach, or other similar model to prepare applicable questions for checking with the user, and process input(s) of the user in response to the questions based on previous records and data (e.g., historical data indicating a corresponding condition for an input) to determine a condition of the user. By considering a conflict resolution proposal from the conflict resolver, the dynamic questioning agent may also be configured to suggest its question(s) in a subsequent round to capture more relevant and complete information. It will be appreciated that the input and data to be processed by the system for determining an urgency level for treatment of a user are obtained from one or more devices, modules, consoles, servers, or other similar hardware that may be configured to provide such input and data with or without interaction with the user. In some embodiments, information derived from the argument set and analysis of patient behavioral or physiological data may be used to generate guidance for the user or patient regarding whether, and how urgently, to seek medical attention. Such guidance may include, for example, advising the patient to call emergency services immediately, to visit a clinic or hospital within a predetermined time window, or to monitor symptoms at home under specified conditions.

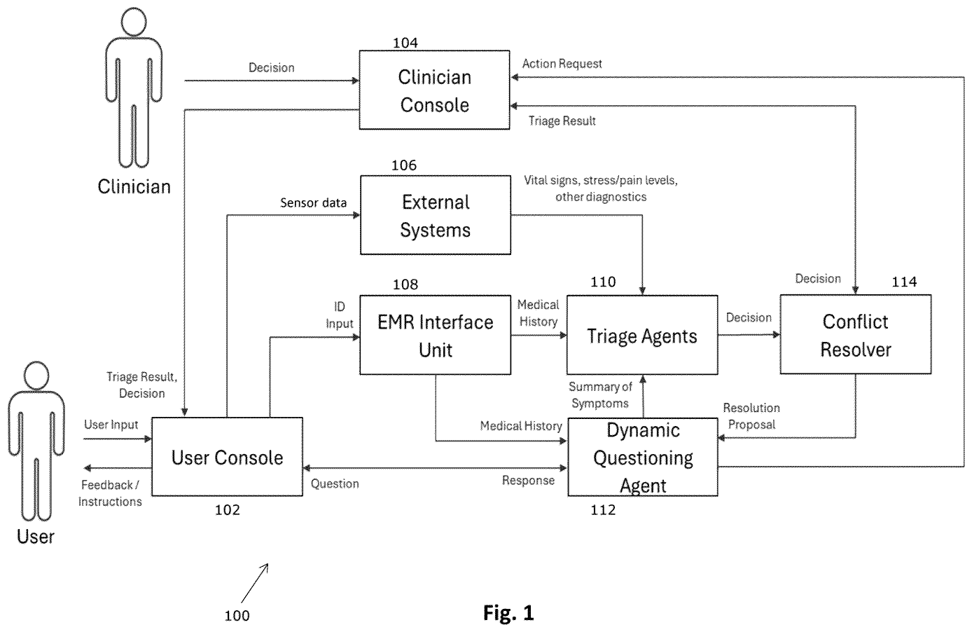

FIG. 1 shows a block diagram for a system 100 for determining an urgency level for treatment of a user according to various embodiments of the present disclosure. For example, the system 100 may comprise a user console 102, a clinician console 104, an external systems module 106, an EMR Interface unit 108, a triage agents module 110, a dynamic questioning agent module 112, and a conflict resolver module 114.

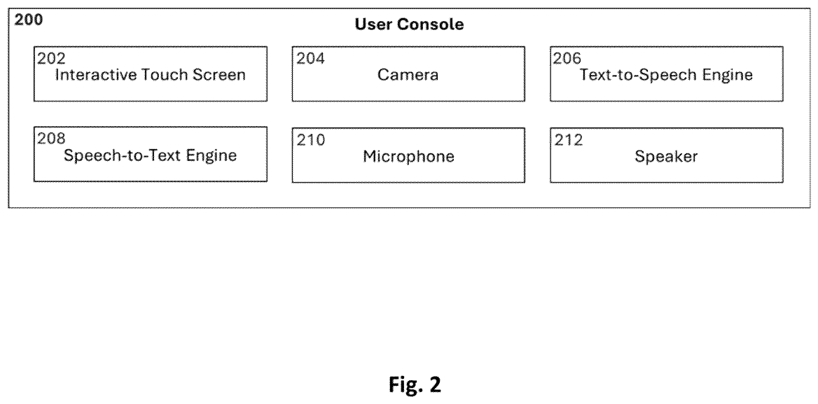

The user console 102 may be configured to interact with a user to assess an urgency level for treatment of the user. Referring to block diagram 200 in FIG. 2 for the user console 102, the user console 102 may comprise an interactive touch screen that may be configured to display feedback, questions, instructions and other similar interactions from the dynamic questioning agent module 112, and allow the user to type or select a response for a question or instruction posed by the dynamic questioning agent module 112. The user console 102 may also comprise a camera 204 configured to capture the user's image (e.g., image of the user's face, hands, body, or other similar image) for analysis to assess the user's condition. The user console 102 may also comprise a text-to-speech engine 206 configured for converting system-generated text into natural-sounding speech for delivering interactive prompts and feedback that may be received from the user, a speech-to-text engine 208 for converting the user's speech into text (e.g., for user's response to the dynamic questioning agent module 112), a microphone 210 for capturing the user's voice (e.g., for the speech-to-text engine 208 and/or for analysis of the user's speech to determine an urgency level), a speaker 212 configured for outputting sound-based feedback and instructions (e.g., from the dynamic questioning agent module 112), and other similar modules. Sensor data such as user's image and voice captured by the camera 204 or the microphone 210 may be sent to the external system 106 for their analysis, and ID information obtained through the user console 102 may be sent to the EMR interface unit 108. In addition, triage result and decision output from the clinician console 104 may be displayed on the screen to interact with the user, or to trigger the clinical console 104 to obtain updated sensor data.

The external systems module 106, with reference to block diagram 300 for the external systems module 106, may comprise a Vital Sign Measurement Unit 302 configured for measuring vital signs including heart rate, blood pressure (systolic and diastolic), respiratory rate, oxygen saturation, temperature, and other vital signs of the user (e.g., based on an image/images captured by the camera 204 of the User Console 102), and may also be configured to output numerical values and corresponding timestamps associated with the measured vital signs. The external systems module 106 may also comprise a Facial Expression Analysis Unit 304 that is configured for analysing facial expressions of the user (e.g., based on an image captured by the camera 204 of the user console 102) to detect stress and pain, or to analyse and determine a status of skin surface or facial parts such as eyes, or to detect specific actions such as vomiting. It may also be configured to output numerical values and corresponding timestamps associated with the analysis. The external systems module 106 may also comprise a Speech Emotion Analysis Unit 306 that may be configured for analysing speech emotions (e.g., based on an audio captured by the microphone 210 of the User Console 102) to detect stress and pain, and may also be configured to output numerical values with corresponding timestamps associated with the analysis. The external systems module 106 may also comprise one or more Diagnostic Units 308 that may be configured for perform medical diagnostic tests and outputting test results in text format with corresponding timestamp for the diagnostic tests. The analysis performed by the external systems, such as vital signs, stress/pain levels, and other diagnostics, may be sent to Triage Agents 110.

The EMR interface unit 108 may be configured for retrieving medical history of the user (e.g., using the user's identity obtained via the user console 102). The retrieved medical history may be sent to the triage agent 110 and the dynamic questioning agent 112.

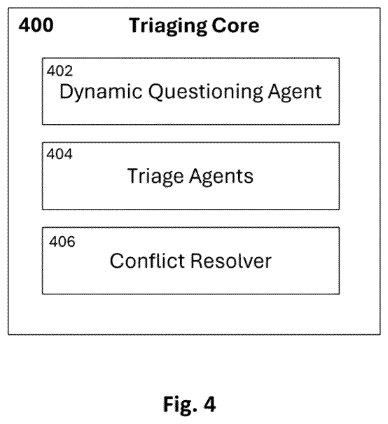

Referring to FIG. 4, the triage agents module 110, a dynamic questioning agent module 112, and a conflict resolver module 114 may be part of a triaging core module 400. The triaging core module 400 may comprise a dynamic questioning agent module 402 (e.g., corresponding to the dynamic questioning agent module 112) that may be configured for using a prompt (e.g., text-based, sound-based, visual-based, for other similar prompt) via the user console 102 to ask questions to gather information about the user's symptoms. In subsequent iterations, it may be configured to use a conflict resolution proposal (e.g., obtained from the conflict resolver module 114) to decide if follow-up questions need to be asked (e.g., generating a question to get information that is able to attack an ‘undecided’ argument, so that an input from the user in response to the generated question may be analysed, a new argument may be generated based on the analysed input, and an argument set may be modified by adding the new argument to the argument set), diagnostics need to be done, clinician needs to provide a triage assessment, or other similar response based on the conflict resolution proposal. The dynamic questioning agent module 402 may also be configured to output an action request and a summary of symptoms to a clinician console 104 and triage agents 110, respectively. The triaging core module 400 may also comprise one or more triage agents 404 (e.g., corresponding to the triage agents module 110) that may each be configured for generating an argument by determining an urgency level and its rationale based on data obtained from either an external system (e.g., via the external systems module 106), an EMR interface unit (e.g., via the EMR interface unit 108), a symptoms summary from a user's answer (e.g., obtained from the dynamic questioning agent 112 based on the input from the user console 102), or other similar sources. A plurality of arguments obtained from the triage agents constitute an argument set. The triaging core module 400 may also comprise a conflict resolver 406 (e.g., corresponding to the conflict resolver module 114) that may be configured to verify if there is any inconsistency in the triage results (e.g., urgency levels) for arguments included in the argument set, and then output a conflict resolution proposal to the dynamic questioning agent 112 based on the verification.

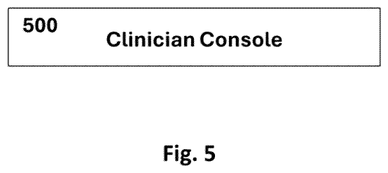

Further, the clinician console 104 (represented as block diagram 500 in FIG. 5) may be configured for displaying verification results obtained from the conflict resolver module 114 and allowing a clinician to input a decision of whether to continue or complete the triage. In an example, the clinician's decision may be sent back to the conflict resolver module 114 as a new argument for further processing of the urgency levels obtained from the triage agents module 110. In another example, the clinician may end the triage process, for example if no conflicts in the urgency levels within the argument set obtained from the triage agents are identified by the conflict resolver module 114, and an urgency level is confirmed for the user. The clinician console 104 may also be configured for receiving an action request from the dynamic questioning agent 112. The clinician can control, based on the action request, the next step of triage via decision output from the clinician console 104 to the user console 102, such as triggering the user console 102 to update physical features, or to include another device such as ECG to measure the user's status for further triage process.

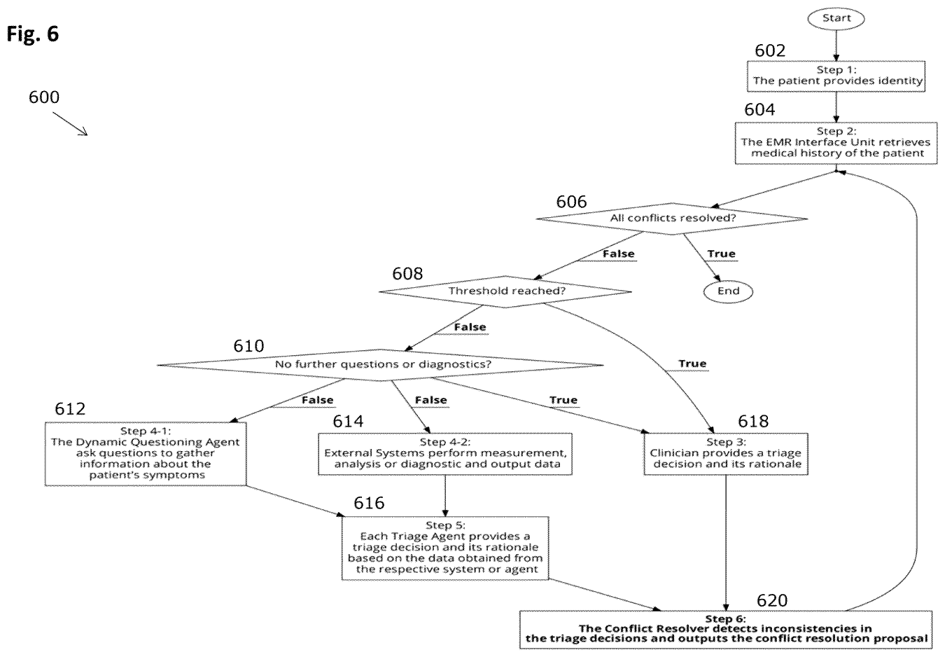

FIG. 6 shows an exemplary flow diagram 600 for determining an urgency level for treatment of a user according to various embodiments of the present disclosure. In step 602, an identity of a user may be provided (e.g., an identity number, passport number, patient number, or other similar form of identification via the user console 102). The identity of the user may also be obtained automatically by a method of biometrics authentication such as face recognition with an image captured by the camera 204, or speaker recognition with a voice captured by the microphone 210. Fingerprint recognition may also be used in a case where a fingerprint scanner is implemented on the interactive touch screen 202. In step 604, the EMR interface unit 108 may be configured to retrieve medical history (e.g., from a database of a medical institution, government body, and/or other similar sources) of the user based on the identity of the user obtained from step 602. In step 606, it is determined if all conflicts (e.g., relating to inconsistency in urgency levels determined by triage agents for the user) are resolved at the conflict resolver 114. If all conflicts are resolved such that an appropriate urgency level has been determined for the user, the process ends. The result is sent to the clinician console 104 with an action request indicating that no action is necessary. If there are any unresolved conflicts, or if no urgency level has been determined at the first time, the process proceeds to step 608 in which it is determined whether a threshold for a number of iteration of triage processes is reached. If the threshold is reached, an action request may be sent to the clinician console 104 together with a triage result at this point, and the process proceeds to step 618 in which a clinician may make a triage decision and its rationale (e.g., a clinician may ‘arbitrate’ the triage decision in a case where the condition in step 608 is true). The decision made by the clinician may be sent back to the conflict resolver 114 and in step 620, the conflict resolver 114 detects any inconsistencies in the triage decisions and outputs a conflict resolution proposal. The process then returns to step 606 for a determination again whether all conflicts are resolved (based on the latest conflict resolution proposal). In an implementation, step 618 may be omitted such that input from a clinician is not required, and the process proceeds from step 608 directly to step 620.

If it is determined in step 608 that the threshold has not been reached, the process proceeds to step 610 in which it is determined, at the conflict resolver 114, whether there are any further questions or diagnostics that need to be asked or performed (e.g., to the user) in order to determine an urgency level or verify any inconsistency among the urgency levels determined by the one or more triage agents. If it is determined that there are no further questions or diagnostics required, an action request may be sent to the clinician console 104 together with a triage result at this point (e.g., a clinician may ‘arbitrate’ the triage decision in a case where the condition in step 610 is true), and the process proceeds to and continues from step 618 as explained above. If it is determined in step 610 that there are further questions to be asked or diagnostics to be performed, a resolution proposal may be sent to the dynamic questioning agent 112, and the process proceeds to step 612 in which the dynamic questioning agent module 112 may be configured to ask, based on the resolution proposal, the further questions to gather information about the user's condition. In addition, the process proceeds to step 614 in which the external systems module 106 may be configured to perform further measurements, analysis and/or diagnostics relating to the user's condition, and then output the results for further review and update (if required) of the previously determined urgency level(s), and/or to add a further urgency level based on the results to further review the previously determined urgency level(s). The step 614 may be done automatically, or by triggering the user console 102 to capture and send updated sensor data to the external system 106 based on the triage result or decision input from the clinician console 104, which may be configured to be controlled automatically or by a clinician based on an action request input from the dynamic agent 112 based on the resolution proposal from the conflict resolver 114. In another case, the conflict resolver 114 may directly trigger the user console 102 to capture updated sensor data. The process then proceeds to step 616 in which each triage agent provides a triage decision and its rationale (e.g., an updated urgency level based on a condition of the user) based on the data obtained from the respective system (e.g., via the external system module 106) or triage agent, which may be configured to be done at the triage agents module 110. The process further proceeds to step 620 and continues as explained above.

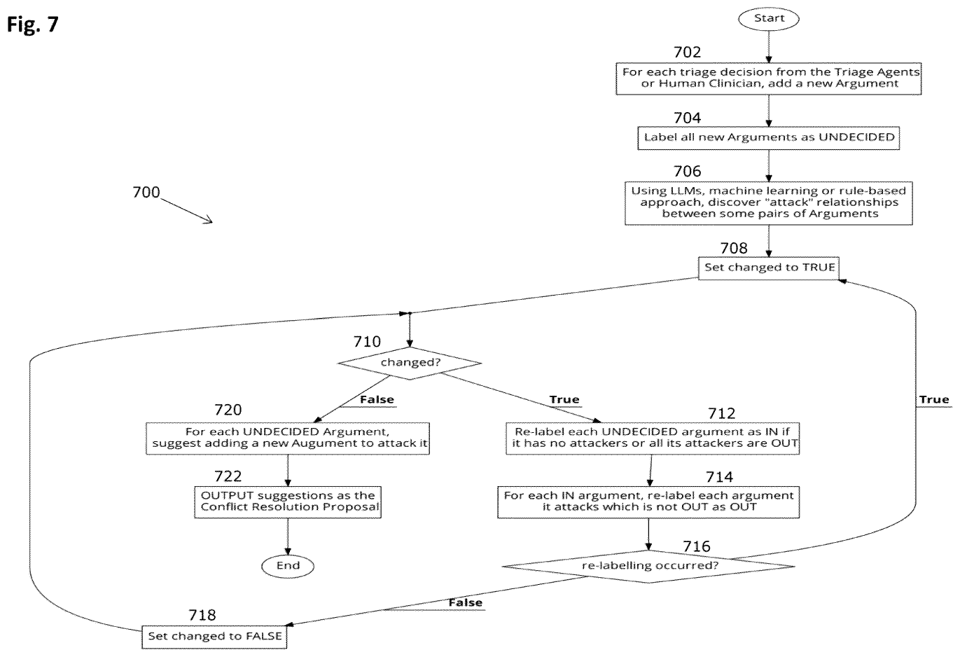

FIG. 7 shows an exemplary flow diagram 700 for how conflicts among arguments with different urgency level results may be resolved in step 620 in FIG. 6 according to various embodiments of the present disclosure. In step 702, a new argument is added for each triage decision (e.g., each urgency level) from one or more triage agents in the triage agents module 110 or a clinician input in the clinician console 104. For example, each urgency level that is determined by a triage agent or a clinician may be represented as an argument, and these arguments are put together to generate an argument set and processed (e.g., processed by a ML model, LLM, AI, rule-based approach, or other similar models based on historical data identifying a corresponding urgency level for a condition) to verify if there are any inconsistencies in each of the urgency levels and their corresponding condition. For example, argument association information refers to information associated with an argument and this may comprise one or more attributes such as a name of the argument, a list of arguments that are attacking this argument (e.g., a list of arguments that may contradict and/or invalidate this argument), a list of arguments that this argument is attacking, a state of this argument (e.g., undecided, in, or out), or other similar attribute. For example, if the state of the argument is ‘undecided’, it may mean that more information or a new argument (e.g., adding a newly determined urgency level/argument associated with a new means to obtain a condition of a user, for further triage processing in order to ‘attack’ an ‘undecided’ argument) is required to verify if there is an inconsistency in an urgency level associated with the argument. If the state of the argument is ‘in’, it may mean that the urgency level associated with the argument is accepted to be credible with its rationale (e.g., credible for the corresponding condition of the user). The credibility may be determined based on historical data identifying a corresponding urgency level for a condition, a ML model, LLM, AI, or a rule-based approach (e.g., based on one or more rules that may be set in the utilized model by a medical institution or expert, a ML model, LLM, AI, or other similar entity). If the state of the argument is ‘out’, it may mean that the urgency level associated with the argument is considered as inconsistent with another argument and therefore may be omitted from further triage processing, or further reviewed and updated based on new information on the condition of the user. The implementation of such arguments enables an improved and more efficient conflict resolution among differing triage results.

In step 704, all new arguments within the argument set are labelled as undecided. In step 706, ‘attack’ relationships between one or more pairs of arguments may be determined utilizing an LLM, ML model, rule-based approach, AI, or other similar models. For example, a first argument and a second argument may be determined based on an urgency level and/or condition associated with the first and second argument, and (in subsequent steps) the first and second argument may be obtained based on historical data indicating a corresponding urgency level for a condition. This advantageously enables an efficient comparison between two urgency levels that may be conflicting so that any inconsistency can be identified and resolved. In step 708, a Boolean variable ‘changed’ is set to ‘true’. In step 710, the Boolean variable ‘changed’ is checked whether it is set to ‘true’ or ‘false’. If the Boolean variable ‘changed’ is ‘true’, the process proceeds to step 712 in which each ‘undecided’ argument is relabelled as ‘in’ if there are no attackers against it, or all its attackers against it are ‘out’. In step 714, each argument that is not labelled as ‘out’ and that is attacked by an ‘in’ argument is relabelled to ‘out’. In step 716, it is determined whether any relabelling has occurred in steps 712 and 714. If it is determined that there was relabelling, the process proceeds back to step 708 in which the Boolean variable ‘changed’ is set to ‘true’. Otherwise, the process proceeds to step 718 in which the Boolean variable ‘changed’ is set to ‘false’, and the process proceeds back to step 710. On the other hand, if the Boolean variable ‘changed’ is ‘false’ in step 710, the process proceeds to step 720 in which a new argument is suggested to be added in the triage process in order to ‘attack’ the ‘undecided’ argument(s) (e.g., a suggestion to add a further urgency level, indicated in a conflict resolution proposal by the conflict resolver module 114, based on a verification of an inconsistency in one or more urgency levels determined by one or more triage agents 404 of the triage agents module 110). Thus, the Boolean variable ‘changed’ is a flag to indicate whether any argument re-labelling has occurred in step 712 and/or step 714. If there is, then this flag is set to true, which necessitates the next iteration. In other words, the program keeps repeating step 712 and 714 until there is no more argument re-labelling. In step 722, based on a further review of the previous and newly added arguments, a further conflict resolution proposal may be generated, for example for review and a final triage decision by a clinician (e.g., determination of an appropriate urgency level for the user in view of the conflict resolution proposal by the clinician) or for recommending an appropriate urgency level for the user, and the process ends.

Triage agents may be configured to determine the triage (of the user) based on patient-related information obtained from external systems, EMR interface units, or past question-and-answer histories about the patient. They may then be configured to output an argument consisting of the triage results and its rationales (e.g., determining an urgency level based on a corresponding condition of a user).

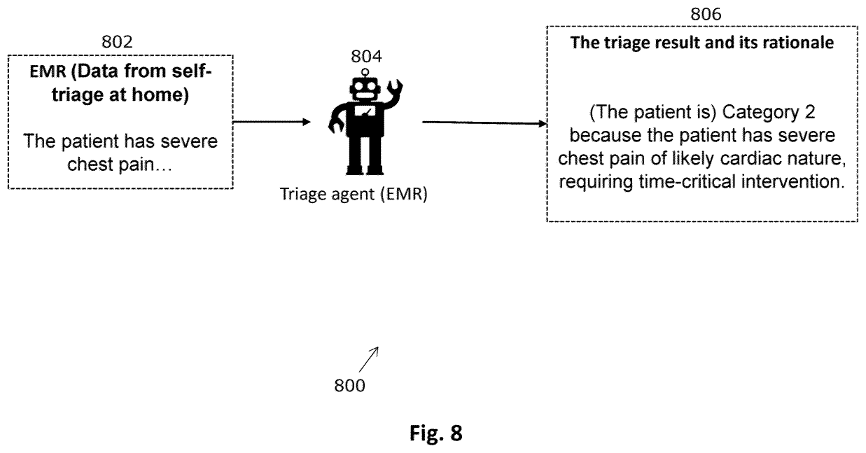

FIG. 8 shows an exemplary illustration 800 of an urgency level based on an electronic medical record (EMR) of a user according to an embodiment of the present disclosure. For example, based on data 802 obtained from an EMR of a user (e.g., in which the data 802 shows that the user has a condition of severe chest pain), triage agent 804 may output an argument 806 in which an urgency level of category 2 is determined with a rationale that the user has severe chest pain of likely cardiac nature and requires time-critical intervention.

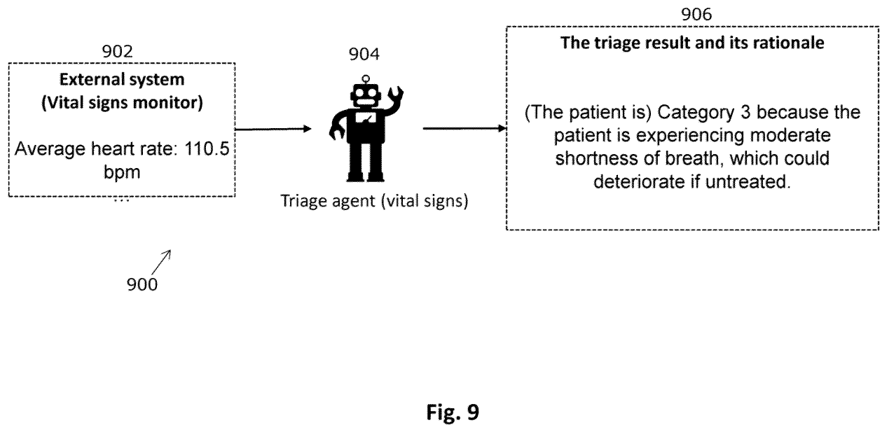

FIG. 9 shows an exemplary illustration 900 of an urgency level based on vital signs monitoring of a user according to an embodiment of the present disclosure. For example, based on data 902 obtained from a vital signs monitor for a user (e.g., in which it is shown that the user has an average heart rate of 110.5 beats per minute (bpm)), triage agent 904 may output an argument 906 in which an urgency level of category 3 is determined with a rationale that the user is experiencing moderate shortness of breath which could deteriorate if untreated.

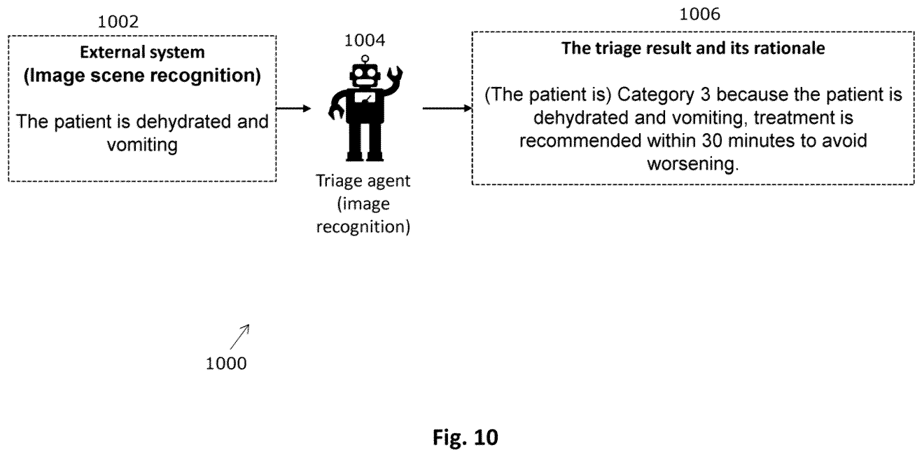

FIG. 10 shows an exemplary illustration 1000 of an urgency level based on image recognition of a user according to an embodiment of the present disclosure. For example, based on data 1002 obtained from an image recognition process (e.g., analysing an image of the user's face captured at the time of the triage process) for the user (e.g., in which the analysed image shows that the user is dehydrated and vomiting), triage agent 1004 may output an argument 1006 in which an urgency level of category 3 is determined with a rationale that the user is dehydrated and vomiting, and treatment is recommended within 30 minutes to avoid worsening.

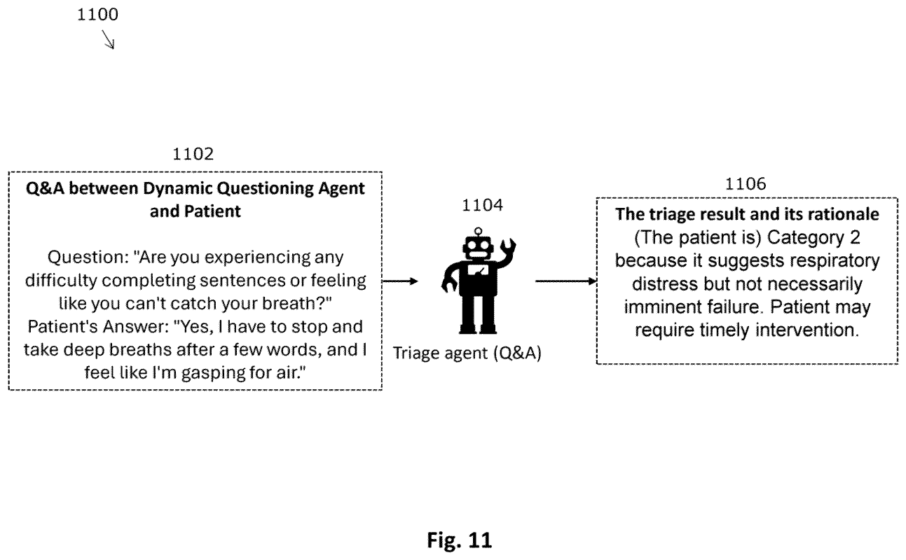

FIG. 11 shows an exemplary illustration 1100 of an urgency level based on a question and answer (Q&A) session between a dynamic questioning agent and a user according to an embodiment of the present disclosure. For example, data 1102 may be obtained from a Q&A session between a dynamic questioning agent (e.g., utilizing the dynamic questioning agent module 112) and the user and analysed by triage agent 1104. For example, the Q&A session may comprise a question “Are you experiencing any difficulty completing sentences or feeling like you can't catch your breath?” to the user, and the user's answer is “Yes, I have to stop and take deep breaths after a few words, and I feel like I'm gasping for air”. Based on this data, the triage agent 1104 may output an argument 1106 in which an urgency level of category 2 is determined with a rationale that the data suggests respiratory distress but not necessarily imminent failure, and that the user may require timely intervention.

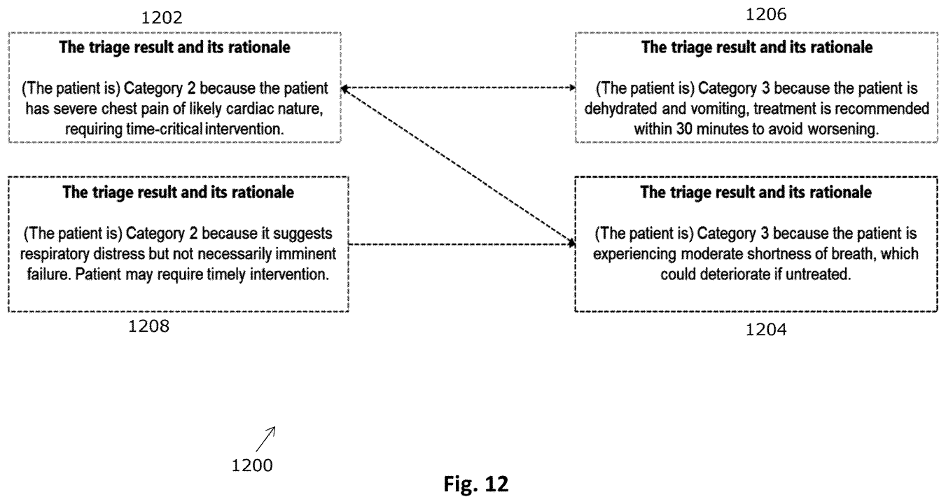

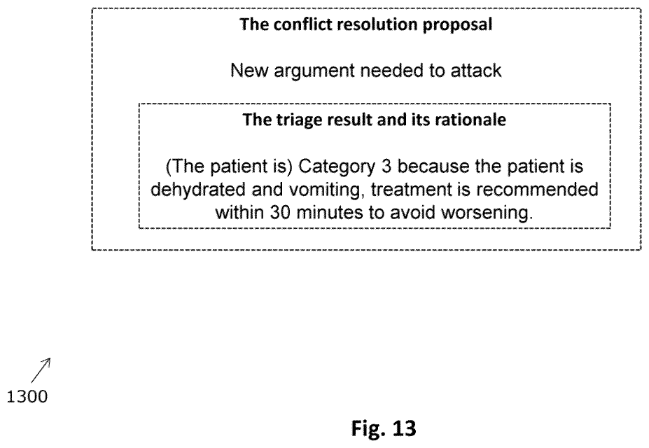

FIG. 12 shows an exemplary illustration 1200 of a conflict resolution process for different urgency level results according to an embodiment of the present disclosure. The determined urgency levels from arguments 806, 906, 1006 and 1106 (e.g., in the form of corresponding arguments 1202, 1204, 1206 and 1208 respectively in an argument set) may be processed by a conflict resolver (e.g., conflict resolver module 114) which detects inconsistencies in the triage results and information relating to the user and/or condition of the user. Each arrow indicates a direction of attack among the arguments. For example, argument 1202 and argument 1206 are under attack from each other, argument 1202 and argument 1204 are under attack from each other, and argument 1204 is also under attack from argument 1208. Then, the conflict resolver outputs a conflict resolution proposal. For example, it may be determined by the conflict resolver module 114 that triage result 1202 is an ‘undecided’ argument, triage result 1204 is an ‘out’ (e.g., rejected) argument, triage result 1206 is an ‘undecided’ argument, and triage result 1208 is an ‘in’ (e.g., accepted) argument. Thus, referring to FIG. 13, the conflict resolver may output a conflict resolution proposal 1300 in which it is suggested that a new argument (e.g., an additional triage result with its rationale) is needed to ‘attack’ the ‘undecided’ argument 1206 e.g., since all attackers against the undecided argument 1206 are ‘undecided’ and not ‘in’ (e.g., argument 1202 is an ‘undecided’ argument).

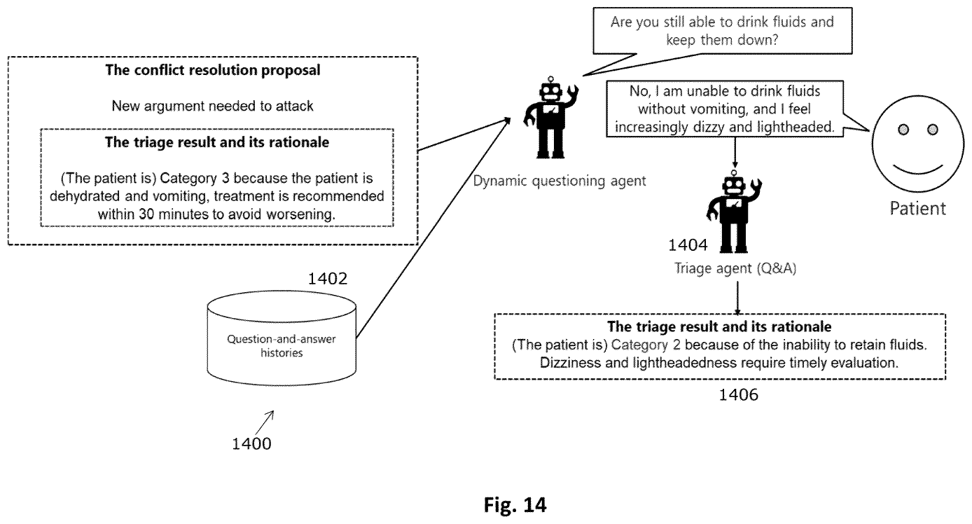

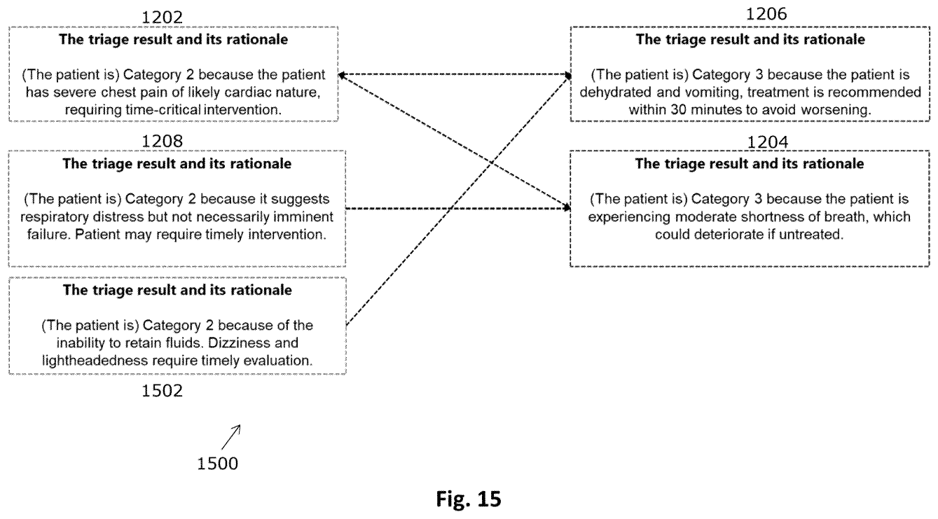

Based on the conflict resolution proposal, information updates and retrievals may be carried out. The dynamic questioning agent 112 may determine the next steps based on the conflict resolution proposal and question-and-answer data (e.g., data obtained from a previous or current Q&A session with the user). For example, referring to illustration 1400 of FIG. 14, data 1402 may be retrieved (e.g., from the dynamic questioning agent) of a question posed to the user “Are you still able to drink fluids and keep them down?” and an answer “No, I am unable to drink fluids without vomiting, and I feel increasingly dizzy and lightheaded” provided by the user. Based on this data, which may be sent from the dynamic questioning agent 112 to a triage agent 1404 within the triage agents module 110, the triage agent 1404 may be configured to output an argument 1406 in which a determined urgency level is category 2 e.g., because of the user's inability to retain fluids, and dizziness and light-headedness require timely evaluation, to add the argument to the argument set as a new argument 1502. Further referring to FIG. 15, the conflict resolver detects inconsistencies in the triage results and patient information based on the previous arguments 1202, 1204, 1206 and 1208 as well as the new argument 1502 (e.g., new argument corresponding to added argument 1406). For example, new argument 1502 and argument 1202 (previously considered as ‘undecided’) are now considered as ‘in’ arguments (e.g., accepted arguments), and argument 1206 is now considered as ‘out’ argument e.g., this argument is rejected. Since all arguments have been classified as either ‘in’ or ‘out’, the conflict among the triage results is considered resolved. Since all triage results that are included in ‘in’ arguments indicate category 2 as an appropriate urgency level, it will be adopted as the overall triage result of the agents. Thus, the conflict resolver outputs a conflict resolution proposal (e.g., proposing category 2 as the overall urgency level) based on the analysis.

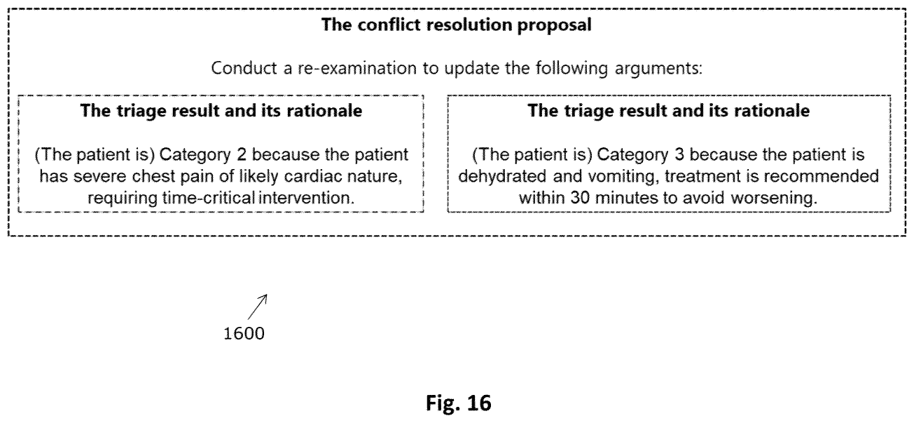

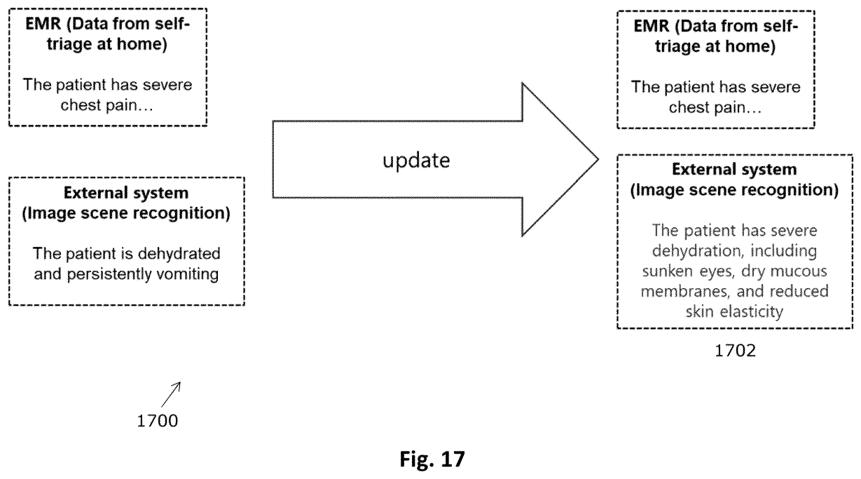

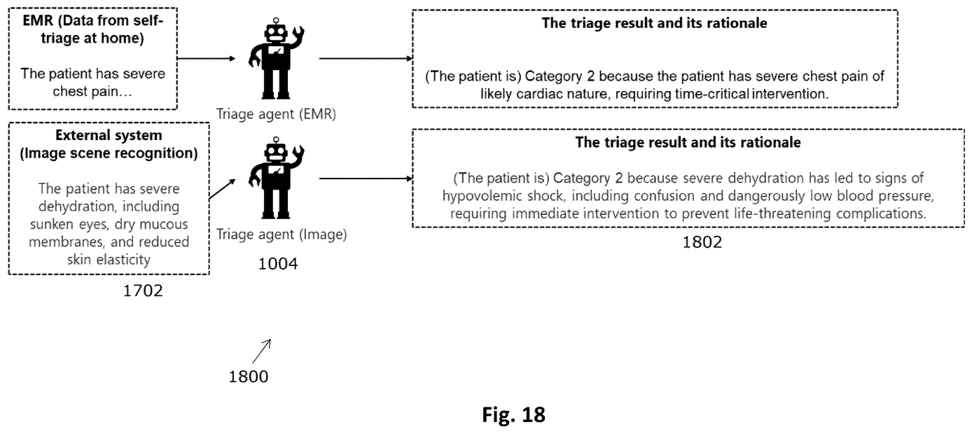

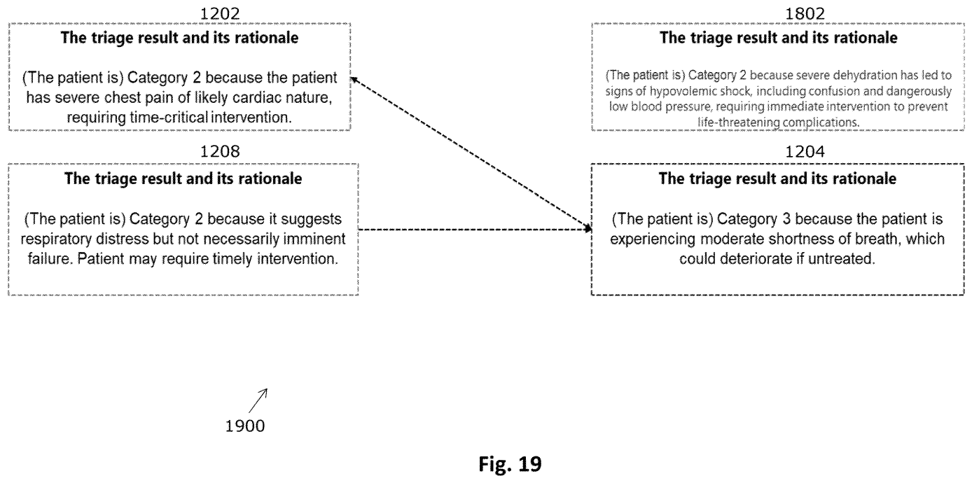

FIG. 16 shows an exemplary illustration 1600 of an alternative conflict resolution proposal according to an embodiment of the present disclosure, in which it is suggested by the conflict resolver to conduct a re-examination to update ‘undecided’ arguments 1202 and 1206. Based on the resolution proposal from the conflict resolver 114, information updates and retrievals are carried out. A clinician may perform information updates and retrievals based on the proposal, which may be sent from the dynamic questioning agent as an action request based on the resolution proposal. In this example, the clinician waits for new information from external systems such as updated EMR or image analysis results. The update may be configured to be done by the clinician's triggering the user console to retrieve and update sensor data or by automatically updating the sensor data in the user console 102. For example, referring to illustration 1700 of FIG. 17, based on an updated image recognition analysis 1702 (e.g., from external systems module 106), it may be determined that the user has severe dehydration, including sunken eyes, dry mucous membranes, and reduced skin elasticity. Thus, further referring to FIG. 18, the triage agent 1004 may be configured to update the previous argument 1006 in the argument set to new argument 1802, determining an urgency level of category 2 because severe dehydration has led to signs of hypovolemic shock, including confusion and dangerously low blood pressure, requiring immediate intervention to prevent life-threatening complications. On the other hand, no changes are made to argument 1202 because there is no change in the EMR data of the user. Thus, based on the previous arguments 1202, 1204 and 1208 as well as updated argument 1802 in the updated argument set as shown in illustration 1900 of FIG. 19, the conflict resolver detects inconsistencies in the triage results and patient information. As a result of the argument 1802 update, the attack relationship with argument 1202 is removed (since both arguments 1202 and 1802 indicate a same urgency level of category 2), and there are no more undecided arguments (since argument 1204 is now rejected). Thus, the conflict resolver outputs a conflict resolution proposal e.g., indicating category 2 as the overall urgency level for treatment of the user.

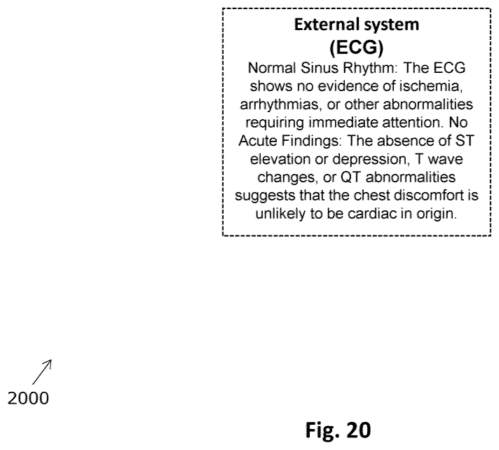

FIG. 20 shows another exemplary illustration 2000 of how the alternative conflict resolution proposal may be responded to according to an embodiment of the present disclosure, in which new data for ECG is obtained (e.g., via the external systems module 106) instead of image recognition data. This may be configured to be done by triggering the user console 102 (e.g., automatically by the conflict resolver 114, a clinician based on a conflict resolution proposal, or other similar entity), which may be configured to retrieve updated ECG data (or other updated sensor data which may be recommended in the conflict resolution proposal e.g., for updating an argument or adding a new argument), and the data may be configured to be sent to the external system 106 for analysis. For example, the new ECG data obtained indicates a normal sinus rhythm, in which the ECG shows no evidence of ischemia, arrhythmias, or other abnormalities requiring immediate attention. Further, there are no acute findings. The absence of ST elevation or depression, T wave changes, or QT abnormalities suggests that the chest discomfort is unlikely to be cardiac in origin.

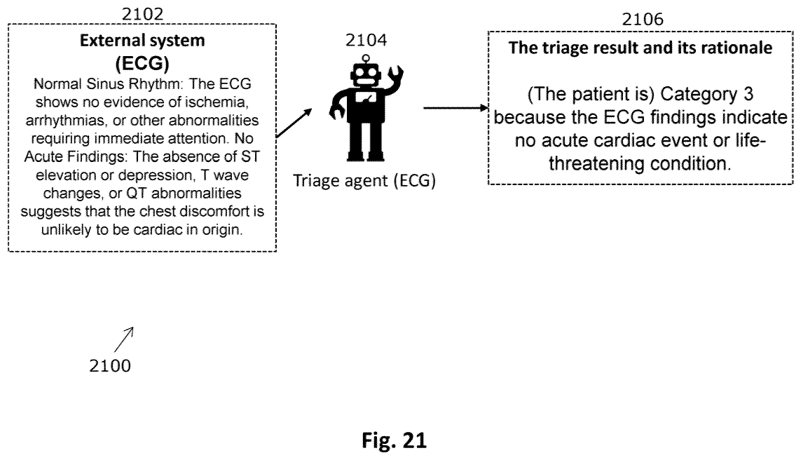

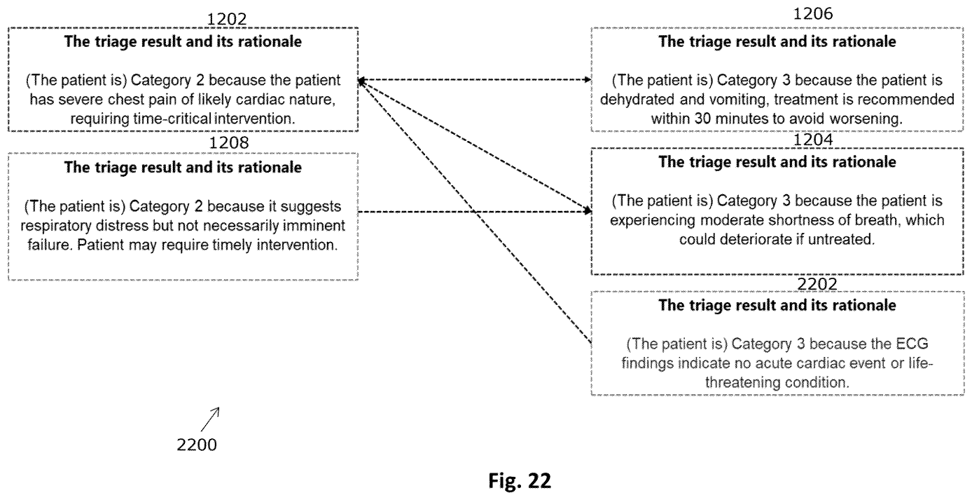

Based on the above data (shown in illustration 2100 of FIG. 21 as data 2102), a new triage agent 2104 for ECG may be configured to generate an argument including a triage result 2106 and its rationale, in which an urgency level of category 3 is determined for the user because the ECG findings indicate no acute cardiac event or life-threatening condition. Thus, referring to illustration 2200 of FIG. 22, previous arguments 1202, 1204, 1206 and 1208 as well as new argument 2202 corresponding to new argument 2106 form a new argument set and are processed by the conflict resolver to detect inconsistencies in the triage results and patient information. For example, as a result of the new argument 2202, argument 1202 indicating an urgency level of category 2 is ‘attacked’ and rejected, and argument 1206 is accepted since the only argument attacking it is argument 1202 which is now rejected. Thus, no more arguments are ‘undecided’, and the conflict resolver thus outputs a conflict resolution proposal based on the analysis.

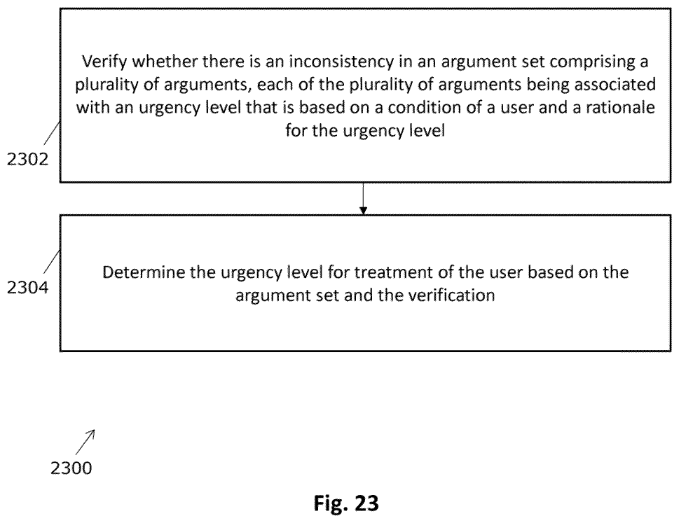

FIG. 23 shows a flow diagram for determining an urgency level for treatment of a user according to various embodiments of the present disclosure. In step 2302, it is verified whether there is an inconsistency in an argument set comprising a plurality of arguments, each of the plurality of arguments being associated with an urgency level that is based on a condition of a user and a rationale for the urgency level. In step 2304, the urgency level for treatment of the user is determined based on the argument set and the verification.

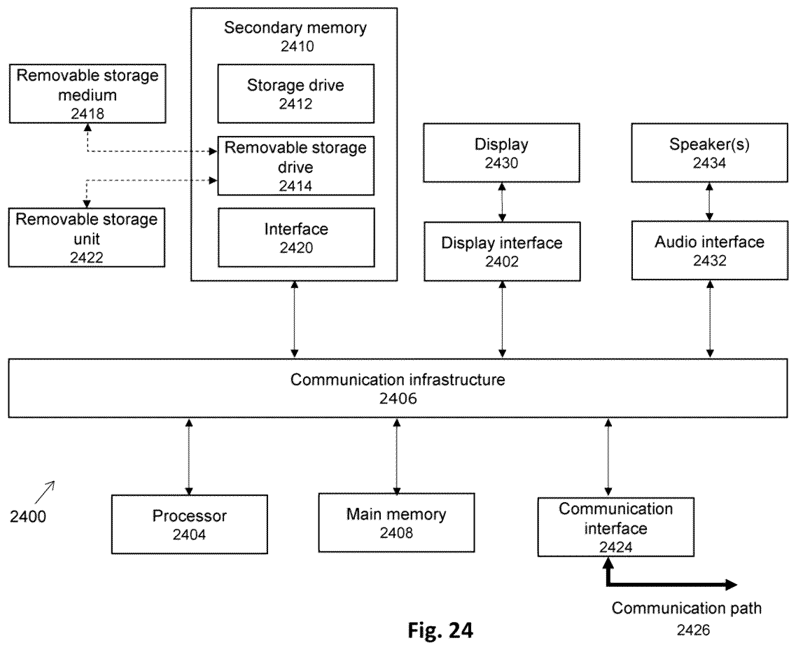

FIG. 24 depicts an exemplary computing device 2400, hereinafter interchangeably referred to as a computer system 2400, where one or more such computing devices 2400 may be used to execute the method of FIGS. 6 to 23. The exemplary computing device 2400 can be used to implement a system for determining an urgency level for treatment of a user. The following description of the computing device 2400 is provided by way of example only and is not intended to be limiting.

As shown in FIG. 24, the example computing device 2400 includes a processor 2404 for executing software routines. Although a single processor is shown for the sake of clarity, the computing device 2400 may also include a multi-processor system. The processor 2404 is connected to a communication infrastructure 2406 for communication with other components of the computing device 2400. The communication infrastructure 2406 may include, for example, a communications bus, cross-bar, or network.

The computing device 2400 further includes a main memory 2408, such as a random access memory (RAM), and a secondary memory 2410. The secondary memory 2410 may include, for example, a storage drive 2412, which may be a hard disk drive, a solid state drive or a hybrid drive and/or a removable storage drive 2414, which may include a magnetic tape drive, an optical disk drive, a solid state storage drive (such as a USB flash drive, a flash memory device, a solid state drive or a memory card), or the like. The removable storage drive 2414 reads from and/or writes to a removable storage medium 2418 in a well-known manner. The removable storage medium 2418 may include magnetic tape, optical disk, non-volatile memory storage medium, or the like, which is read by and written to by removable storage drive 2414. As will be appreciated by persons skilled in the relevant art(s), the removable storage medium 2418 includes a computer readable storage medium having stored therein computer executable program code instructions and/or data.

In an alternative implementation, the secondary memory 2410 may additionally or alternatively include other similar means for allowing computer programs or other instructions to be loaded into the computing device 2400. Such means can include, for example, a removable storage unit 2422 and an interface 2420. Examples of a removable storage unit 2422 and interface 2420 include a program cartridge and cartridge interface (such as that found in video game console devices), a removable memory chip (such as an EPROM or PROM) and associated socket, a removable solid state storage drive (such as a USB flash drive, a flash memory device, a solid state drive or a memory card), and other removable storage units 2422 and interfaces 2420 which allow software and data to be transferred from the removable storage unit 2422 to the computer system 2400.

The computing device 2400 also includes at least one communication interface 2424. The communication interface 2424 allows software and data to be transferred between computing device 2400 and external devices via a communication path 2426. In various embodiments of the disclosure, the communication interface 2424 permits data to be transferred between the computing device 2400 and a data communication network, such as a public data or private data communication network. The communication interface 2424 may be used to exchange data between different computing devices 2400 which such computing devices 2400 form part an interconnected computer network. Examples of a communication interface 2424 can include a modem, a network interface (such as an Ethernet card), a communication port (such as a serial, parallel, printer, GPIB, IEEE 1394, RJ45, USB), an antenna with associated circuitry and the like. The communication interface 2424 may be wired or may be wireless. Software and data transferred via the communication interface 2424 are in the form of signals which can be electronic, electromagnetic, optical or other signals capable of being received by communication interface 2424. These signals are provided to the communication interface via the communication path 2426.

As shown in FIG. 24, the computing device 2400 further includes a display interface 2402 which performs operations for rendering images or videos to an associated display 2430 and an audio interface 2432 for performing operations for playing audio content via associated speaker(s) 2434.

As used herein, the term “computer program product” may refer, in part, to removable storage medium 2418, removable storage unit 2422, a hard disk installed in storage drive 2412, or a carrier wave carrying software over communication path 2426 (wireless link or cable) to communication interface 2424. Computer readable storage media refers to any non-transitory, non-volatile tangible storage medium that provides recorded instructions and/or data to the computing device 2400 for execution and/or processing. Examples of such storage media include magnetic tape, CD-ROM, DVD, Blu-ray Disc, a hard disk drive, a ROM or integrated circuit, a solid state storage drive (such as a USB flash drive, a flash memory device, a solid state drive or a memory card), a hybrid drive, a magneto-optical disk, or a computer readable card such as a PCMCIA card and the like, whether or not such devices are internal or external of the computing device 2400. Examples of transitory or non-tangible computer readable transmission media that may also participate in the provision of software, application programs, instructions and/or data to the computing device 2400 include radio or infra-red transmission channels as well as a network connection to another computer or networked device, and the Internet or Intranets including e-mail transmissions and information recorded on Websites and the like.

The computer programs (also called computer program code) are stored in main memory 2408 and/or secondary memory 2410. Computer programs can also be received via the communication interface 2424. Such computer programs, in a case where they are executed, enable the computing device 2400 to perform one or more features of embodiments discussed herein. In various embodiments, the computer programs, in a case where they are executed, enable the processor 2404 to perform features of the above-described embodiments. Accordingly, such computer programs represent controllers of the computer system 2400.

Software may be stored in a computer program product and loaded into the computing device 2400 using the removable storage drive 2414, the storage drive 2412, or the interface 2420. The computer program product may be a non-transitory computer readable medium. Alternatively, the computer program product may be downloaded to the computer system 2400 over the communications path 2426. The software, in a case where it is executed by the processor 2404, causes the computing device 2400 to perform the necessary operations to execute the method as shown in FIGS. 6 to 23.

It is to be understood that the embodiment of FIG. 24 is presented merely by way of example to explain the operation and structure of a system for determining an urgency level for treatment of a user. Therefore, in some embodiments one or more features of the computing device 2400 may be omitted. Also, in some embodiments, one or more features of the computing device 2400 may be combined together. Additionally, in some embodiments, one or more features of the computing device 2400 may be split into one or more component parts.

It will be appreciated by a person skilled in the art that numerous variations and/or modifications may be made to the present disclosure as shown in the specific embodiments without departing from the spirit or scope of the disclosure as broadly described. The present embodiments are, therefore, to be considered in all respects to be illustrative and not restrictive.

Further, the whole or part of the embodiments disclosed above can be described as, but not limited to, the following supplementary notes.

### (Supplementary Note 1)

A method for determining an urgency level for treatment of a user, including:
verifying, by a processor, whether there is an inconsistency in an argument set including a plurality of arguments, each of the plurality of arguments being associated with an urgency level that is based on a condition of a user and a rationale for the urgency level; and
determining, by the processor, the urgency level for treatment of the user based on the argument set and the verification.

### (Supplementary Note 2)

The method of Supplementary Note 1, in which verifying whether there is an inconsistency includes processing the plurality of arguments included in the argument set based on historical data indicating a corresponding urgency level for a condition.

### (Supplementary Note 3)

The method of Supplementary Notes 1 or 2, in which the condition is obtained by processing an input of the user in response to a question in a question and answer (Q&A) session with the user, or by capturing sensor data with at least one of a camera or a microphone and analysing the sensor data; and a determination of the urgency level based on the condition of the user is conducted by a triage agent.

### (Supplementary Note 4)

The method of Supplementary Note 3, in which the Q&A session is a dynamically generated query session based on at least one of a past urgency level, previously obtained sensor data and EMR-based medical history.

### (Supplementary Note 5)

The method of Supplementary Notes 3 to 4, in which analysing the sensor data includes extracting at least one of a vital sign, a facial expression, speech emotion, and ECG of the user.

### (Supplementary Note 6)

The method of Supplementary Notes 1 to 5, in which verifying whether there is an inconsistency includes processing at least one attack among the plurality of arguments that have different urgency levels to find an inconsistency.

### (Supplementary Note 7)

The method of Supplementary Note 6, in which verifying whether there is an inconsistency includes labeling each of the plurality of arguments included in the argument set based on the attack with one of ‘in’, ‘out’, and ‘undecided’, generating a conflict resolution proposal that reduces a number of ‘undecided’ arguments; and modifying the argument set based on the conflict resolution proposal to reduce inconsistency; in which ‘in’ is a label for a credible argument against which no unrejected attack exist, ‘out’ is a label for an argument attacked by at least one ‘in’ argument, and ‘undecided’ is a label for an argument neither ‘in’ nor ‘out’.

### (Supplementary Note 8)

The method of Supplementary Note 7, in which credibility of an argument is determined based on the historical data, a large language model, or a rule-based approach.

### (Supplementary Note 9)

The method of Supplementary Notes 7 to 8, in which the conflict resolution proposal instructs to perform at least one of a dynamic questioning that generates a new question to reduce the number of ‘undecided’ arguments, an argument update that updates status of an argument by updating the sensor data, and a new means addition that adds a new means to generate a new argument.

### (Supplementary Note 10)

The method of Supplementary Note 9, further including generating a question to get information that is able to attack an ‘undecided’ argument, analysing an input from the user in response to the generated question, generating a new argument based on the analysed input, and modifying the argument set by adding the new argument to the argument set.

### (Supplementary Note 11)

The method of Supplementary Note 9, in which the argument update includes retrieving updated sensor data, analysing the updated sensor data to obtain an updated condition of the user, and updating a status of an ‘undecided’ argument based on the updated condition.

### (Supplementary Note 12)

The method of Supplementary Note 9, in which the new means addition includes generating a new argument associated with a new means to obtain a condition of the user, and modifying the argument set by adding the new argument to the argument set.

### (Supplementary Note 13)

The method of Supplementary Note 12, in which the new means includes an electrocardiogram (ECG).

### (Supplementary Note 14)

A system for determining an urgency level for treatment of a user, including:
at least one processor; and
at least one memory including computer program code; the at least one memory and the computer program code configured to, with at least one processor, cause the system at least to:
verify whether there is an inconsistency in an argument set including a plurality of arguments, each of the plurality of arguments being associated with an urgency level that is based on a condition of a user and a rationale for the urgency level; and
determine the urgency level for treatment of the user based on the argument set and the verification.

### (Supplementary Note 15)

The system of Supplementary Note 14, in which verifying whether there is an inconsistency includes processing the plurality of arguments included in the argument set based on historical data indicating a corresponding urgency level for a condition.

### (Supplementary Note 16)

The system of Supplementary Notes 14 or 15, in which the condition is obtained by processing an input of the user in response to a question in a question and answer (Q&A) session with the user, or by capturing sensor data with at least one of a camera or a microphone and analysing the sensor data; and a determination of the urgency level based on the condition of the user is conducted by a triage agent.

### (Supplementary Note 17)

The system of Supplementary Note 16, in which the Q&A session is a dynamically generated query session based on at least one of a past urgency level, previously obtained sensor data and EMR-based medical history.

### (Supplementary Note 18)

The system of Supplementary Notes 16 to 17, in which analysing the sensor data includes extracting at least one of a vital sign, a facial expression, speech emotion, and ECG of the user.

### (Supplementary Note 19)

The system of Supplementary Notes 14 to 16, in which verifying whether there is an inconsistency includes processing at least one attack among the plurality of arguments that have different urgency levels to find an inconsistency.

### (Supplementary Note 20)

The system of Supplementary Note 19, in which verifying whether there is an inconsistency includes labeling each of the plurality of arguments included in the argument set based on the attack with one of ‘in’, ‘out’, and ‘undecided’, generating a conflict resolution proposal that reduces a number of ‘undecided’ arguments; and modifying the argument set based on the conflict resolution proposal to reduce inconsistency; in which ‘in’ is a label for a credible argument against which no unrejected attack exist, ‘out’ is a label for an argument attacked by at least one ‘in’ argument, and ‘undecided’ is a label for an argument neither ‘in’ nor ‘out’.

### (Supplementary Note 21)

The system of Supplementary Note 20, in which credibility of an argument is determined based on the historical data, a large language model, or a rule-based approach.

### (Supplementary Note 22)

The system of Supplementary Notes 20 to 21, in which the conflict resolution proposal instructs to perform at least one of a dynamic questioning that generates a new question to reduce the number of ‘undecided’ arguments, an argument update that updates status of an argument by updating the sensor data, and a new means addition that adds a new means to generate a new argument.

### (Supplementary Note 23)

The system of Supplementary Note 22, further configured to generate a question to get information that is able to attack an ‘undecided’ argument, analyse an input from the user in response to the generated question, generate a new argument based on the analysed input, and modify the argument set by adding the new argument to the argument set.

### (Supplementary Note 24)

The system of Supplementary Note 22, in which the argument update includes retrieving updated sensor data, analysing the updated sensor data to obtain an updated condition of the user, and updating a status of an ‘undecided’ argument based on the updated condition.

### (Supplementary Note 25)

The system of Supplementary Note 22, in which the new means addition includes generating a new argument associated with a new means to obtain a condition of the user, and modifying the argument set by adding the new argument to the argument set.

### (Supplementary Note 26)

The system of Supplementary Note 25, in which the new means includes an electrocardiogram (ECG).

### (Supplementary Note 27)

The method according to Supplementary Note 1, in which information derived from the argument set and a result of machine learning-based analysis of patient behavioral or physiological data are used to generate guidance supporting decision making by the patient regarding whether to seek medical attention.

### (Supplementary Note 28)

The system according to Supplementary Note 14, in which information derived from the argument set and a result of machine learning-based analysis of patient behavioral or physiological data are used to generate guidance supporting decision making by the patient regarding whether to seek medical attention.

What is claimed is:

1. A computer-implemented method for determining an urgency level for treatment of a user, comprising:
obtaining, by at least one processor from a plurality of triage agents, patient-related information for the user including at least one of electronic medical record data retrieved via an electronic medical record (EMR) interface unit, vital sign data, and image analysis data obtained via an external systems module, and generating, by the plurality of triage agents, a plurality of arguments each including an urgency level that is based on a condition of the user and a rationale for the urgency level, the plurality of arguments constituting an argument set;
verifying, by a conflict resolver executed by the at least one processor, whether there is an inconsistency in the argument set, wherein verifying comprises:
(i) initially labeling each of the plurality of arguments as an undecided argument;
(ii) determining, by using historical data indicating corresponding urgency levels for conditions and at least one of a machine learning model, a large language model, and a rule-based approach, attack relationships between pairs of the plurality of arguments having different urgency levels;
(iii) iteratively relabeling each undecided argument as an in argument when there is no attacker against the undecided argument or all attackers against the undecided argument are out arguments, and relabeling each argument that is not an out argument and that is attacked by an in argument as an out argument, until a Boolean variable indicating whether any relabeling has occurred indicates that no further relabeling has occurred; and
(iv) when at least one undecided argument remains in the argument set after the iteratively relabeling, generating a conflict resolution proposal including at least one of a further question to be asked to the user by a dynamic questioning agent and an instruction to obtain updated sensor data for the user via a user console or to include a new device for measuring a condition of the user, and updating the argument set based on at least one new or updated argument generated in response to the conflict resolution proposal; and
determining, by the conflict resolver executed by the at least one processor, the urgency level for treatment of the user as an overall urgency level based on arguments in the argument set that are labeled as in arguments after the verifying.

2. The computer-implemented method of claim 1, wherein
verifying whether there is an inconsistency comprises processing the argument set based on historical data indicating a corresponding urgency level for a condition, the historical data being retrieved via the EMR interface unit from an electronic medical record of the user and stored in at least one memory.

3. The computer-implemented method of claim 1, wherein
the condition is obtained by processing an input of the user in response to a question in a question and answer (Q&A) session with the user presented via the user console, or by capturing sensor data with at least one of a camera or a microphone connected to an external systems module and analyzing the sensor data; and
a determination of the urgency level based on the condition of the user is conducted by a triage agent implemented as part of a triage agents module executed by the at least one processor.

4. The computer-implemented method of claim 3, wherein
the Q&A session is a dynamically generated query session performed by a dynamic questioning agent module based on at least one of a past urgency level, previously obtained sensor data and EMR-based medical history retrieved from an electronic medical record via the EMR interface unit.

5. The computer-implemented method of claim 3, wherein
analyzing the sensor data by the external systems module comprises extracting at least one of a vital sign, a facial expression, speech emotion, and ECG of the user.

6. The computer-implemented method of claim 1, wherein
verifying whether there is an inconsistency comprises evaluating, by a conflict resolver module, at least one attack among the plurality of arguments that have different urgency levels to find an inconsistency.

7. The computer-implemented method of claim 6, wherein
verifying whether there is an inconsistency comprises performing a labeling step in which the plurality of arguments are labeled as ‘in’, ‘out’ or ‘undecided’, wherein:
‘in’ is assigned to credible arguments against which no unrejected attack exists;
‘out’ is assigned to arguments that are attacked by at least one argument labeled as ‘in’; and
‘undecided’ is a label for an argument neither ‘in’ nor ‘out’ and corresponds to an undecided argument in the iterative relabeling performed by the conflict resolver.

8. The computer-implemented method of claim 7, wherein
credibility of an argument is evaluated by the conflict resolver by using historical data, a large language model, or a rule-based approach, and attack relationships between the plurality of arguments are determined based on the evaluated credibility.

9. The computer-implemented method of claim 7, wherein
the conflict resolution proposal comprises at least one of:
a dynamic questioning that asks a further question to the user to generate a new argument;
an argument update that updates an argument based on updated condition information; and
a device addition that generates a new argument by including an additional device in communication with the external systems module.

10. The computer-implemented method of claim 9, further comprising:
generating, by the dynamic questioning agent module, a question to be asked to the user in accordance with the dynamic questioning;
presenting the question via the user console;
acquiring an answer from the user; and
updating the argument set by adding the new argument to the argument set.

11. The computer-implemented method of claim 9, wherein the argument update comprises:
obtaining updated condition information for the user by at least one of re-running the Q&A session and obtaining updated sensor data via the external systems module or a clinician console; and
updating an urgency label or the status of an ‘undecided’ argument based on the updated condition information.

12. The computer-implemented method of claim 9, wherein the device addition comprises:
including a new device for measuring the condition of the user via the external systems module or a clinician console; and
updating the argument set by adding the new argument to the argument set.

13. The computer-implemented method of claim 12, wherein
the device includes an electrocardiogram (ECG) measurement obtained from an ECG device connected to the external systems module.

14. A system for determining an urgency level for treatment of a user, comprising:
at least one processor;
at least one memory including computer program code;
a user console;
an external systems module;
an EMR interface unit;
a triage agents module;
a dynamic questioning agent module; and
a conflict resolver module,
the at least one memory and the computer program code being configured to, with the at least one processor, cause the system at least to:
obtain, via the external systems module and the EMR interface unit, patient-related information for the user including at least one of electronic medical record data, vital sign data, and image analysis data;
generate, by the triage agents module, an argument set comprising a plurality of arguments, each of the plurality of arguments being associated with an urgency level that is based on a condition of the user and a rationale for the urgency level;
verify, by the conflict resolver module, whether there is an inconsistency in the argument set; and
determine the urgency level for treatment of the user as an overall urgency level based on the argument set and the verification.

15. The system of claim 14, wherein verifying whether there is an inconsistency comprises:
processing the argument set based on historical data indicating a corresponding urgency level for a condition stored in the at least one memory.

16. The system of claim 14, wherein
the condition is obtained by processing an input of the user in response to a question in a question and answer (Q&A) session with the user presented via the user console, or by capturing sensor data with at least one of a camera or a microphone connected to the external systems module and analyzing the sensor data; and
a determination of the urgency level based on the condition of the user is conducted by a triage agent of the triage agents module.

17. The system of claim 16, wherein
the Q&A session is a dynamically generated query session performed by the dynamic questioning agent module based on at least one of a past urgency level, previously obtained sensor data and EMR-based medical history retrieved via the EMR interface unit.

18. The system of claim 16, wherein
analyzing the sensor data by the external systems module comprises extracting at least one of a vital sign, a facial expression, speech emotion, and ECG of the user.

19. The system of claim 14, wherein
verifying whether there is an inconsistency comprises evaluating, by the conflict resolver module, at least one attack among the plurality of arguments that have different urgency levels to find an inconsistency.

20. The computer-implemented method according to claim 1, wherein
information derived from both the arguments and machine-learning analysis of user behavioral or physiological time series obtained via the external systems module is used to provide guidance output via the user console for decision making by the user regarding whether to seek medical attention.
